(19) United States
(12) Patent Application Publication
(10) Pub. No.: US 2021/0318208 A1
(43) Pub. Date: Oct. 14, 2021

(54) Method of Detection of Diseases Related to Coronavirus Infection by Breath Analysis

(71) Applicant: Hygieia Sciences LLC, Johns Creek, GA (US)

(72) Inventors: Charlene Warres Bayer, Marietta, GA (US); William Hiller, Alpharetta, GA (US)

(73) Assignee: Hygieia Sciences LLC, Johns Creek, GA (US)

(21) Appl. No.: 16/844,180

(22) Filed: Apr. 9, 2020

(51) Int. Cl. G01N 1/22; G01N 33/497; G01N 30/72; H01J 49/26; G01N 30/86; H01J 49/00

(52) U.S. Cl. CPC G01N 1/22; G01N 33/497; G01N 30/7206; G01N 2001/2244; G01N 30/8686; H01J 49/0036; H01J 49/26

(57) ABSTRACT

Analysis of a breath sample is a noninvasive point-of-care tool with ever increasing screening and clinical applicability. Herein we describe a method to analyze the content of low-level, trace volatile organic compounds from alveolar breath captured as a breath sample from a patient suspected of having a disease related to coronavirus infection. The breath sample is then analyzed using a gas phase analysis methodology, such as gas chromatography-mass spectroscopy (“GCMS”), to generate an analysis result, such as a GCMS spectrum. A computer system is then used to develop a fingerprint pattern from the analysis result. The fingerprint pattern is then used to determine a patient status for the patient. The fingerprint pattern is typically a grouping of 75 to 450 compounds of known concentration which are indicative of a particular disease related to coronavirus.

### BACKGROUND

### Field of the Invention

The present invention relates generally to analysis methods for the assessment of breath samples. More specifically, the present invention relates to analysis methods for the evaluation of breath volatile organic compounds (BVOC) within a breath sample to detect diseases related to coronavirus infection within a patient.

### Description of the Related Art

Acceptance of BVOC analysis as clinical screening technique has been slow to develop. This is due to a number of issues including: unsuccessful attempts to find individual or a small number of low-level volatile organic compounds detected in breath as marker compounds for individual diseases; lack of understanding of the physiological meaning of the detected volatiles; a dearth of standardized methods and comparable results among a group of laboratories; the difficulty to adapt sophisticated technology and instrumentation for widespread acceptance in clinical settings; few larger population studies having been conducted; and inability to translate the methods and technology to clinical settings.

Some success has been achieved for tuberculosis, oxidative stress, ulcers, some lung cancer, diabetes, and tracking heart transplant rejection, but success of BVOC analysis in the clinical space has largely been limited due to the above factors. Successful screening for breast cancer has been achieved with high accuracy and precision. Most research in this area is focused on disease detection with only a few BVOCs as the marker.

### SUMMARY

In accordance with the embodiments herein, a method for detection of diseases related to coronavirus infection using breath analysis is disclosed. The method described herein generally utilizes chemical analysis of low-level BVOCs from alveolar breath captured as a breath sample from a patient. The breath sample is then analyzed using a gas phase analysis methodology to generate an analysis result, such as a gas chromatographic chromatogram. A computer system is then used to develop a fingerprint pattern from the analysis result. The fingerprint pattern is then used to determine a patient status related to a specific disease related to coronavirus infection for the patient.

### BRIEF DESCRIPTION OF THE DRAWINGS

FIG. 1 provides a general overview of the method herein described.

### DETAILED DESCRIPTION OF EMBODIMENTS

In the following description, for purposes of explanation and not limitation, details and descriptions are set forth in order to provide a thorough understanding of the present invention. However, it will be apparent to those skilled in the art that the present invention may be practiced in other embodiments that depart from these details and descriptions without departing from the spirit and scope of the invention.

For the purpose of this disclosure, patient status includes diagnosis of inflammatory status, disease state, disease severity, disease progression, therapy efficacy, changes in patient status over time, and monitoring organ and tissue damage from coronavirus infection. Other patient statuses are contemplated.

For the purpose of definition, coronavirus is one or more of the viruses from the coronaviridae family of enveloped, positive-sense, single-strand RNA viruses.

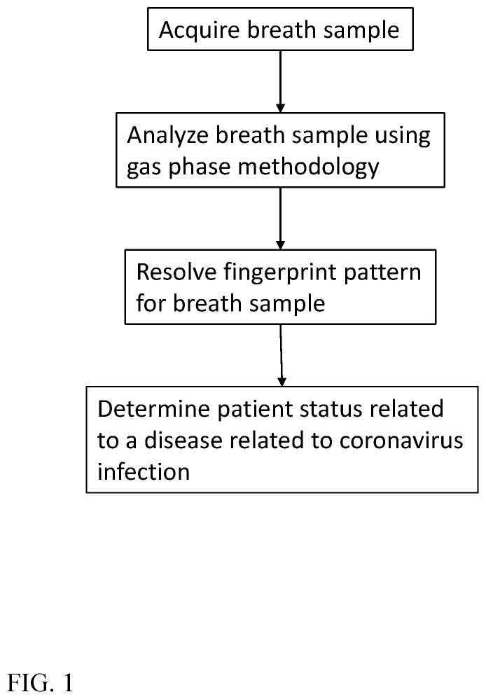

In an illustrative embodiment of the invention, as summarized in FIG. 1, the method may be summarized in the following four steps: i) collecting a breath sample from a patient in a sterile sampling vessel; ii) analyzing the breath sample using a gas phase analysis methodology to generate an analysis result; using a computer system configured to iii) resolving a fingerprint pattern from the analysis result; and iv) determining a patient status for the patient related to a disease related to coronavirus infection based on the fingerprint pattern. Typically, the fingerprint pattern will be 75 to 450 different compounds within the analysis result. It is understood, however, that smaller or larger combinations may be used as a fingerprint pattern.

In a further embodiment, the sterile sampling vessel may contain a carbon dioxide sensor. This sensor could be used to determine when the patient has reached alveolar breath. This would ensure that the breath sample contains low-level VOCs. There are a number of commercially available BVOC collection samplers available which are suitable for the present invention. Many BVOC collection samplers contain a solid-state adsorbent or thin film adsorbent media to adsorb low-level VOCs thus ensuring capture. If a solid-state adsorbent or thin film adsorbent media is used in the breath sampler, thermal desorption or thin film adsorbent media techniques are used to release the low-level VOCs from the solid-state adsorbent prior to analysis with the gas phase analysis methodology.

In a further embodiment, the gas phase analysis methodology could be gas chromatography, mass spectrometry, or gas chromatography-mass spectrometry. Other gas phase analysis methods are contemplated.

In another embodiment, the fingerprint pattern is a spectrographic or chromatographic pattern within the analysis result that is specific to a particular disease related to coronavirus infection. The fingerprint pattern is typically a group of marker compounds between 75 and 450 unique compounds in predetermined concentrations for each specific disease related to coronavirus. It is contemplated, however, that a single or fewer than 75 marker compound(s) could be used to identify a particular disease related to coronavirus infection.

In an additional embodiment, patient status can be inflammatory status, disease state, disease severity, disease progression, efficacy of a particular therapy, changes in patient status over time, or monitoring tissue damage from coronavirus infection. Other patient statuses are contemplated.

In a further embodiment, a disease related to coronavirus infection may be severe acute respiratory syndrome, middle east respiratory syndrome, coronavirus disease 2019, or other diseases related to coronavirus infection. Additionally, organ and tissue damage related to coronavirus infections is also included.

What is claimed is:

1. A method comprising:
collecting a breath sample from a patient in a sterile sampling vessel;
analyzing the breath sample using at least one gas phase analysis methodology to generate an analysis result; and
using a computer system:
resolving a fingerprint pattern from the analysis result; and
determining a patient status related to a disease related to coronavirus infection for the patient based on the fingerprint pattern.

2. The method of claim 1, wherein the sterile sampling vessel contains a carbon dioxide sensor.

3. The method of claim 1, wherein the sterile sampling vessel is a commercially available breath sampler.

4. The method of claim 1, wherein the sterile sampling vessel includes an absorbent material selected from the group consisting of solid-state adsorbent and thin film adsorbent media.

5. The method of claim 4, further comprising using thermal desorption techniques to release the breath sample from the absorbent material.

6. The method of claim 1, wherein the at least one gas phase analysis methodology is selected from the group consisting of gas chromatography, mass spectrometry, and gas chromatography-mass spectrometry.

7. The method of claim 1, wherein the fingerprint pattern is a chromatographic pattern specific to a particular disease related to coronavirus.

8. The method of claim 1, wherein the fingerprint pattern is a spectrographic pattern specific to a particular disease related to coronavirus.

9. The method of claim 1, wherein the fingerprint pattern contains at least one marker compound.

10. The method of claim 9, wherein the marker compound is a compound specific to a particular disease related to coronavirus.

11. The method of claim 1, wherein the patient status is selected from the group consisting of inflammatory status, disease state, disease severity, disease progression, therapy efficacy, changes in patient status over time, monitoring organ damage from coronavirus infection, and monitoring tissue damage from coronavirus infection.

12. The method of claim 1, wherein the disease related to coronavirus infection is selected from the group consisting of severe acute respiratory syndrome, middle east respiratory syndrome, and coronavirus disease 2019, organ damage related to coronavirus infection, and tissue damage related to coronavirus infection.
